(19) United States
(12) Patent Application Publication
(10) Pub. No.: US 2007/0022546 A1
(43) Pub. Date: Feb. 1, 2007

(54) 3-Amino-2-aminomethylphenol derivatives and colorants comprising these compounds

(76) Inventors: Gisela Umbricht, Marly (CH); Franco Jose Rosato, Liebefeld (CH); Hans-Jurgen Braun, Ueberstorf (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/351,152

(22) Filed: Feb. 9, 2006

(30) Foreign Application Priority Data
Feb. 9, 2005 (DE) 102005005875.2

(51) Int. Cl. A61K 8/00

(52) U.S. Cl. 8/405

(57) ABSTRACT

3-Amino-2-aminomethylphenol derivatives of the general formula (I) or physiologically compatible, water-soluble salts thereof, 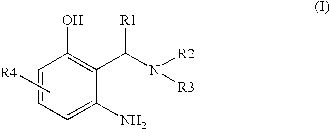 and agent comprising these compounds for the oxidative coloring of keratin fibers.

The invention relates to agents for the coloring of keratin fibers based on a developer substance-coupler substance combination which comprise 3-amino-2-aminomethylphenol derivatives as coupler substance, and to novel 3-amino-2-aminomethylphenol derivatives.

In the field of coloring keratin fibers, in particular hair coloring, oxidation dyes have achieved significant importance. The coloration arises here as a result of the reaction of certain developer substances with certain coupler substances in the presence of a suitable oxidizing agent. The developer substances used here are, in particular, 2,5-diaminotoluene, 2,5-diaminophenylethyl alcohol, p-aminophenol and 1,4-diaminobenzene, while examples of coupler substances are resorcinol, 4-chlororesorcinol, 1-naphthol, 3-aminophenol and derivatives of m-phenylenediamine.

Besides coloring to the desired intensity, numerous additional requirements are placed on oxidation dyes which are used for coloring human hair. For example, the dyes must be acceptable from a toxicological and dermatological point of view and the hair colorations achieved must have good light fastness, permanent wave fastness, acid fastness and rubbing fastness. However, in any case, such colorations must remain stable over a period of at least 4 to 6 weeks without being affected by light, rubbing and chemical agents. Furthermore, it is required that, by combining suitable developer substances and coupler substances, a broad palette of different color nuances can be produced.

Using the currently used colorants, as are described, for example, in the monograph by K. H. Schrader “Grundlagen und Rezepturen der Kosmetika” [Fundamentals and formulations of cosmetics], 2nd edition (1989), pages 784-799, it is, however, not possible to satisfy the abovementioned requirements in all aspects. There therefore continues to be a need for novel developer substances which satisfy the abovementioned requirements to a particular degree.

In this regard, it has now surprisingly been found that certain 3-amino-2-aminomethylphenol derivatives according to the general formula (I) satisfy the requirements placed on coupler substances to a particularly high degree. Thus, when these coupler substances are used with most known developer substances, color-rich color nuances are obtained which are extraordinarily light fast and wash fast.

The present invention therefore provides an agent for the oxidative coloring of keratin fibers, such as, for example, hair, furs, feathers or wool, in particular human hair, based on a developer substance-coupler substance combination which comprises, as coupler substance, at least one 3-amino-2-aminomethylphenol derivative of the formula (I) or its physiologically compatible, water-soluble salt, 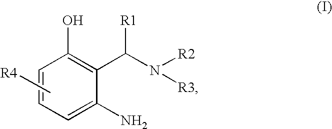
in which
R1 is hydrogen or a C₁-C₆-alkyl group;
R2 and R3, independently of one another, are hydrogen, a saturated C₁-C₆-alkyl group, an unsaturated C₂-C₆-alkyl group, a C₂-C₄-hydroxyalkyl group, a C₃-C₄-dihydroxyalkyl group, a C₂-C₄-aminoalkyl group, a C₁-C₄-dimethylaminoalkyl group, a C₂-C₄-acetylaminoalkyl group, a C₁-C₄-methoxyalkyl group, a C₁-C₄-ethoxyalkyl group, a C₁-C₄-cyanoalkyl group, a C₁-C₄-carboxyalkyl group, a C₁-C₄-aminocarbonylalkyl group, a pyridylmethyl group, a furfuryl group, a hydrogenated furfuryl group, a substituted pyridyl group or a radical of the formula (II), (III) or (IV), 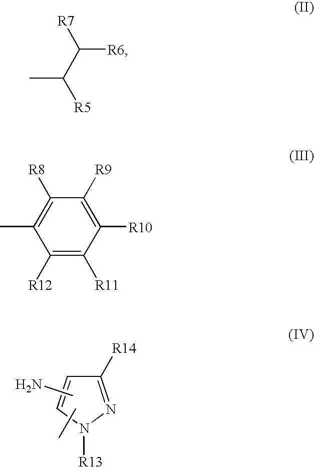
or R2 and R3, together with the nitrogen atom, form a heterocyclic ring of the formula; 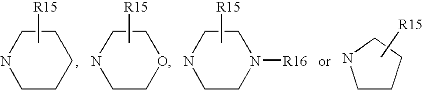
R4 is hydrogen, a halogen atom, a C₁-C₄-alkyl group, a C₁-C₄-hydroxyalkyl group or a C₁-C₄-alkoxy group;
R5 is hydrogen, a carboxyl group, or an aminocarbonyl group;
R6 and R7, independently of one another, are hydrogen, a hydroxyl group, an aminocarbonyl group, a methylthiomethyl group, a phenyl radical substituted by a phenyl group or hydroxyl group or a radical of the formula 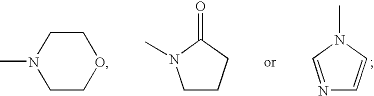
R8, R9, R10, R11 and R12, independently of one another, are hydrogen, a halogen atom, a cyano group, a hydroxyl group, a C₁-C₄-alkoxy group, a C₁-C₄-hydroxyalkoxy group, a C₁-C₆-alkyl group, a C₁-C₄-alkyl thioether group, a mercapto group, a nitro group, an amino group, an alkylamino group, a hydroxyalkylamino group, a dialkylamino group, a di(hydroxyalkyl)amino group, a (dihydroxyalkyl)amino group, a (hydroxyalkyl)alkylamino group, a trifluoromethane group, a —C(O)H— group, a —C(O)CH₃— group, a —C(O)CF₃— group, a —Si(CH₃)₃— group, a C₁-C₄-hydroxyalkyl group or a C₃-C₄-dihydroxyalkyl group or two adjacent radicals R8 to R12 form an —O—CH2-O— bridge;
R13 is a C₁-C₄-alkyl group or a C₁-C₄-hydroxyalkyl group;
R14 is hydrogen or a C₁-C₆-alkyl group;
R15 is one or more hydrogen atoms, hydroxyl groups, carboxyl groups, aminocarbonyl groups or hydroxymethyl groups;
R16 is hydrogen or a C₁-C₆-alkyl group; where at least one of the radicals R2 and R3 is different from hydrogen.

Compounds of the formula (I) which may be mentioned are, for example, the following compounds:
3-amino-2-[(2,3-dihydroxypropyl)aminomethyl]phenol; 3-amino-2-(ethylaminomethyl)phenol; 3-amino-2-(isopropylaminomethyl)phenol; 3-amino-2-(propylaminomethyl)phenol; 3-amino-2-(pyrrolidin-1-ylmethyl)phenol; 3-amino-2-{[(2-furylmethyl)amino]methyl}phenol; 3-amino-2-[(2-methoxy-ethylamino)methyl]phenol; 3-amino-2-(4-morpholinylmethyl)phenol; 3-amino-2-(2-hydroxy-6-aminobenzylamino)butan-1-ol; 3-amino-2-{[(furan-2-ylmethyl)amino]methyl}phenol; 3-amino-2-(4-methylpiperazin-1-ylmethyl)phenol; 1-(2-hydroxy-6-aminobenzyl)piperidin-4-ol; N-[2-(2-hydroxy-6-aminobenzylamino)ethyl]acetamide; 3-amino-2-[(2-morpholin-4-yl-ethylamino)methyl]phenol; 3-amino-2-(allylaminomethyl)phenol; 2-(2-hydroxy-6-aminobenzylamino)propan-1-ol; 3-amino-2-[(3-imidazol-1-ylpropylamino)methyl]phenol; 3-amino-2-(1-piperidinylmethyl)phenol; 3-amino-2-{[(tetrahydrofuran-2-ylmethyl)amino]methyl}phenol; 3-amino-2-((4-aminophenyl)aminomethyl)phenol; 3-(2-hydroxy-6-aminobenzylamino)phenol; 5-(2-hydroxy-6-aminobenzylamino)-2-methylphenol; 3-amino-2-[(2-dimethylaminoethylamino)methyl]phenol; 4-(2-hydroxy-6-aminobenzylamino)butan-1-ol; 3-amino-2-[(3-ethoxypropylamino)methyl]phenol; 3-amino-2-[(3-methoxyphenylamino)methyl]phenol; 3-amino-2-[(4-chlorophenylamino)methyl]phenol; 3-amino-2[(cyclopropylmethylamino)methyl]phenol; 2-(2-hydroxy-6-aminobenzylamino)-4-nitrophenol; 3-amino-2-[(4-chloro-benzylamino)methyl]phenol; 2-[(2-hydroxy-6-aminobenzyl)methylamino]ethanol; 2-[(2-hydroxy-6-aminobenzyl)ethylamino]ethanol; 3-amino-2-{[(pyridin-4-ylmethyl)amino]methyl}phenol; 1-[3-(2-hydroxy-6-aminobenzylamino)propyl]pyrrolidin-2-one; 3-amino-2-(4-amino-2-methylphenyl)aminomethylphenol; 3-amino-2-(4-amino-3-methylphenyl)aminomethylphenol; 2-[5-amino-2-(2-hydroxy-6-aminobenzylamino)phenyl]ethanol; 3-amino-2-(3-aminophenyl)aminomethylphenol; 4-[2-(2-hydroxy-6-aminobenzylamino)ethyl]benzenesulfonamide; 2-[4-amino-2-(2-hydroxy-6-aminobenzylamino)phenoxy]ethanol; 2-[(2-hydroxy-6-aminobenzyl)(2-hydroxyethyl)amino]ethanol; [1-(6-amino-2-hydroxybenzyl)pyrrolidin-2-yl]methanol; 1-(6-amino-2-hydroxybenzyl)pyrrolidin-3-ol; 1-(6-amino-2-hydroxybenzyl)pyrrolidine-2-carboxamide; 1-(6-amino-2-hydroxybenzyl)piperidin-3-ol; 2-(6-amino-2-hydroxybenzylamino)propane-1,3-diol; 2-(6-amino-2-hydroxybenzylamino)-3-hydroxypropionamide; 2-(6-amino-2-hydroxybenzylamino)succinic acid; 3-amino-2-cyclopropylaminomethylphenol; 2-(6-amino-2-hydroxybenzylamino)ethanol; (6-amino-2-hydroxybenzylamino)acetic acid; 4-(6-amino-2-hydroxybenzylamino)phenol; 3-amino-2-(benzo[1,3]dioxol-5-ylaminomethyl)phenol; [(6-amino-2-hydroxybenzyl)methylamino]acetonitrile; 3-amino-2-pentylaminomethylphenol; 3-amino-2-[(3-dimethylaminopropylamino)methyl]phenol; 3-amino-2-{[2-(5-nitropyridin-2-ylamino)ethylamino]methyl}phenol; 3-amino-2-[(2-aminoethylamino)methyl]phenol; 3-[2-(6-amino-2-hydroxybenzylamino)-1-hydroxyethyl]phenol; 3-amino-2-[(4-methylpyridin-2-ylamino)methyl]phenol; 2-(6-amino-2-hydroxybenzyl)-1-methyl -1,2,3,4-tetrahydroisoquinoline-6,7-diol; 2-(2-hydroxy-6-aminobenzylamino)-4-methylsulfanylbutyric acid; 1-(2-hydroxy-6-aminobenzyl)pyrrolidine-2-carboxylic acid; 3-amino-2-(1-pyrrolidinylmethyl)phenol; 3-amino-2-phenylaminomethylphenol; 3-amino-2-(4-dimethylaminophenyl)aminomethylphenol; 1-[3-(6-amino-2-hydroxybenzylamino)phenyl]ethanol; 1-[4-(6-amino-2-hydroxybenzylamino)phenyl]ethanol; 1-[2-(6-amino-2-hydroxybenzylamino)phenyl]ethanol; 3-amino-2-[(3,4-dimethoxyphenylamino)methyl]phenol; 3-amino-2-[(3-fluoro-2-methoxyphenylamino)methyl]phenol; 4-chloro-2-(6-amino-2-hydroxybenzylamino)phenol; 3-amino-2-[(4-trifluoromethylphenylamino)methyl]phenol; 3-amino-2-(p-tolylaminomethyl)phenol; 3-amino-2-[(2-aminophenylamino)methyl]phenol; 3-amino-2-[(2-hydroxyphenylamino)methyl]phenol; 3-amino-2-[(2-dimethylaminophenylamino)methyl]phenol; 3-amino-2-[(2-methylphenylamino)methyl]phenol; 3-amino-2-[(2-trifluoromethylphenylamino)methyl]phenol; 3-amino-2-[(3-dimethylaminophenylamino)methyl]phenol; 3-amino-2-[(3-methylphenylamino)methyl]phenol; 3-amino-2-[(3-trifluoromethylphenylamino]methyl)phenol; 3-amino-2-[(2-bromophenylamino)methyl]phenol; 3-amino-2-[(2-cyanophenylamino)methyl]phenol; 3-amino-2-[(2-fluorophenylamino)methyl]phenol; 3-amino-2-[(2-methoxyphenylamino)methyl]phenol; 3-amino-2-[(2-nitrophenylamino)methyl]phenol; 3-amino-2-[(3-bromophenylamino)methyl]phenol; 3-amino-2-[(3-cyanophenylamino)methyl]phenol; 3-amino-2-[(3-fluorophenylamino)methyl]phenol; 3-amino-2-[(3-methoxyphenylamino)methyl]phenol; 3-amino-2-[(3-nitrophenylamino)methyl]phenol; 3-amino-2-[(4-bromophenylamino)methyl]phenol; 3-amino-2-[(4-cyanophenylamino)methyl]phenol; 3-amino-2-[(4-fluorophenylamino)methyl]phenol; 3-amino-2-[(4-methoxyphenylamino)methyl]phenol; 3-amino-2-[(4-nitrophenylamino)methyl]phenol; 3-amino-2-{[2-(1,3-dihydroxypropyl)aminophenylamino]methyl}phenol; 3-amino-2-{[2-di(2-hydroxyethyl)aminophenylamino]methyl}phenol; 3-amino-2-{[2-(2-hydroxyethylamino)phenylamino]methyl}phenol; 3-amino-2-[(2-pyrrolidinephenylamino)methyl]phenol; 3-amino-2-{[3-(1,3-dihydroxypropyl)aminophenylamino]methyl}phenol; 3-amino-2-{[3-di(2-hydroxyethyl)aminophenylamino]methyl}phenol; 3-amino-2-{[3-(2-hydroxyethyl-amino)phenylamino]methyl}phenol; 3-amino-2-[(3-pyrrolidinephenylamino)methyl]phenol; 3-amino-2-[(4-(1,3-dihydroxypropyl)aminophenylamino)methyl]phenol; 3-amino-2-{[4-di(2-hydroxyethylamino)phenylamino]methyl}phenol; 3-amino-2-{[4-(2-hydroxyethylamino)-phenylamino]methyl}phenol; 3-amino-2-[(4-pyrrolidinephenylamino)methyl]phenol; 3-amino-2-{[4-amino-2-(2-hydroxyethoxy)phenylamino]methyl}phenol; 3-amino-2-[(4-amino-2-chlorophenylamino)methyl]phenol; 3-amino-2-[(4-amino-2-hydroxyphenylamino)methyl]phenol; 3-amino-2-[(4-amino-2-methoxyphenylamino)methyl]phenol; 3-amino-2-{[4-amino-3-(2-hydroxyethoxy)phenylamino]methyl}phenol; 3-amino-2-[(4-amino-3-chloro-phenylamino)methyl]phenol; 3-amino-2-[(4-amino-3-hydroxyphenylamino)methyl]phenol; 3-amino-2-[(4-amino-3-methoxyphenylamino)methyl]phenol; 3-amino-2-[(3,4-diaminophenylamino)methyl]phenol; 3-amino-2-[(2,4-diaminophenylamino)methyl]phenol and 2-[5-amino-4-(2-hydroxy-6-aminobenzylamino)pyrazol-1-yl]ethanol.

Preference is given to compounds of the formula (I) in which (i) R1 and/or R4 is hydrogen, and/or (ii) R2 is a methyl group or a C₁-C₄-hydroxyalkyl group and R3 is a C₁-C₄-hydroxyalkyl group and/or (iii) R2 is hydrogen and R3 is a C₁-C₄-hydroxyalkyl group, a substituted pyridyl radical, a furfuryl radical, a hydrogenated furfuryl radical, a substituted phenyl radical, a substituted pyrazoyl radical or a radical of the following formula 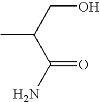
and/or (iv) R2 and R3 form an aliphatic ring of the formula

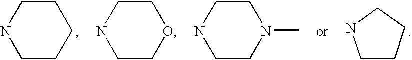

Particular preference is given to the following compounds of the formula (I):
3-amino-2-[(2,3-dihydroxypropyl)aminomethyl]phenol; 3-amino-2-[(2-aminoethylamino)methyl]phenol; 3-amino-2-[(2-hydroxyethylamino)methyl]phenol; 3-amino-2-(4-morpholinylmethyl)phenol; 2-[(2-hydroxy-6-aminobenzyl)-methylamino]ethanol; 3-amino-2-(1-pyrrolidinylmethyl)phenol, 3-amino-2-{[(2-furylmethyl)amino]methyl}phenol, 2-(2-hydroxy-6-aminobenzylamino)propan-1-ol; 2-[(2-hydroxy-6-aminobenzyl)(2-hydroxyethyl)amino]ethanol; [1-(2-hydroxy-6-aminobenzyl)pyrrolidin-2-yl]methanol; 1-(2-hydroxy-6-aminobenzyl)pyrrolidine-2-carboxamide; 3-amino-2-[(4-methylpyridin-2-ylamino)methyl]phenol; 3-amino-2-(1-piperidinylmethyl)phenol, 3-amino-2-[(4-methyl-1-piperazinyl)methyl]phenol, 3-amino-2-((2-aminophenylamino)methyl)phenol; 3-amino-2-((2-chloro-phenylamino)-methyl)phenol; 3-amino-2-[(2-dimethylaminophenylamino)methyl]phenol; 3-amino-2-[(2-fluorophenylamino)methyl]phenol; 3-amino-2-{[2-(2-hydroxyethylamino)phenylamino]methyl}phenol; 3-amino-2-{[2-N,N-bis(2-hydroxyethyl)aminophenylamino]methyl}phenol; 3-amino-2-[(2-pyrrolidinephenylamino)methyl)phenol; 3-amino-2-((3-aminophenylamino)methyl)phenol; 3-amino-2-[(3-chlorophenylamino)methyl]phenol; 3-amino-2-[(3-dimethylaminophenylamino)methyl]phenol; 3-amino-2-[(3-fluorophenyl-amino)methyl]phenol; 3-amino-2-{[3-(2-hydroxyethyl-amino)phenylamino]methyl}phenol; 3-amino-2-{[3-N,N-bis(2-hydroxyethyl)aminophenylamino]methyl}phenol; 3-amino-2-[(3-pyrrolidinephenylamino)methyl}phenol; 3-amino-2-[(4-aminophenylamino)methyl]-phenol; 3-amino-2-((4-chlorophenylamino)methyl)phenol; 3-amino-2-[(4-dimethylaminophenylamino)methyl]phenol; 3-amino-2-[(4-fluorophenylamino)methyl]phenol; 3-amino-2-{[4-(2-hydroxyethylamino)phenylamino]methyl}phenol; 3-amino-2-{[4-N,N-bis(2-hydroxyethyl)aminophenylamino]methyl}phenol; 3-amino-2-[(4-pyrrolidinephenylamino)methyl]phenol; 3-amino-2-{[2-(2-hydroxyethoxy)-4-aminophenylamino]methyl}phenol; 3-amino-2-[(2,4-diaminophenyl-amino)methyl]phenol; 3-amino-2-[(2-chloro-4-aminophenylamino)methyl]phenol; 3-amino-2-[(4-amino-2-hydroxyphenylamino)methyl]phenol; 3-amino-2-[(2-hydroxyethylamino-4-aminophenylamino)methyl]phenol; 3-amino-2-[(2-methyl-4-aminophenylamino)methyl]phenol; 3-amino-2-{[3-(2-hydroxyethoxy)-4-aminophenylamino]methyl}phenol; 3-amino-2-[(3-amino-4-aminophenylamino)-methyl]phenol; 3-amino-2-[(3-chloro-4-aminophenylamino)methyl]phenol; 3-amino-2-[(3-hydroxy-4-aminophenylamino)methyl]phenol; 3-amino-2-{[3-(2-hydroxyethylamino)-4-aminophenylamino]methyl}phenol; 3-amino-2-[(3-methyl-4-aminophenylamino)methyl]phenol; 3-amino-2-{[2-(2-hydroxyethoxy)phenylamino]methyl}phenol; 3-amino-2-[(2-hydroxyphenylamino)methyl}phenol; 3-amino-2-{[3-(2-hydroxyethoxy)phenylamino]methyl}phenol; 3-amino-2-[(3-hydroxyphenylamino)methyl]phenol; 3-amino-2-{[4-(2-hydroxyethoxy)phenylamino]methyl}phenol; 3-amino-2-[(4-hydroxyphenylamino)methyl}phenol; 3-amino-2-(phenylaminomethyl)phenol and 2-[5-amino-4-(2-hydroxy-6-aminobenzylamino)pyrazol-1-yl]ethanol.

The compounds of the formula (I) can be used either in the form of free bases or else in the form of their physiologically compatible salts with inorganic or organic acids, such as, for example, hydrochloric acid, sulfuric acid, phosphoric acid, acetic acid, propionic acid, lactic acid or citric acid.

The 3-amino-2-aminomethylphenol derivatives of the formula (I) are present in the colorant according to the invention in a total amount of from about 0.005 to 20 percent by weight, preference being given to an amount of from about 0.01 to 8 percent by weight and in particular 0.1 to 5 percent by weight.

Suitable developer substances are preferably 1,4-diaminobenzene (p-phenylenediamine), 1,4-diamino-2-methylbenzene (p-tolylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-((diethylamino)methyl)thiophene, 2-chloro-3-(2,5-diaminophenyl)thiophene, 1,4-diamino-2-(pyridin-3-yl)benzene, 2,5-diaminobiphenyl, 2,5-diamino-4′-(1-methylethyl)-1,1′-biphenyl, 2,3′,5-triamino-1,1′-biphenyl, 1,4-diamino-2-methoxymethylbenzene, 1,4-diamino-2-aminomethylbenzene, 1,4-diamino-2-((phenylamino)methyl)benzene, 1,4-diamino-2-((ethyl-(2-hydroxyethyl)amino)methyl)benzene, 1,4-diamino-2-hydroxymethylbenzene, 1,4-diamino-2-(2-hydroxyethoxy)benzene, 2-(2-(acetylamino)ethoxy)-1,4-diaminobenzene, 4-phenylaminoaniline, 4-dimethylamino-aniline, 4-diethylaminoaniline, 4-dipropylaminoaniline, 4-[ethyl(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 4-(((4-aminophenyl)methyl)amino)aniline, 4-[(4-aminophenylamino)methyl]phenol, 1,4-diamino-N-(4-pyrrolidin-1-yl-benzyl)benzene, 1,3-dihydroxy-2-((2-furylmethyl)aminomethyl)benzene, 1,4-diamino-N-thiophen-2-ylmethylbenzene, 1,4-diamino-N-furan-2-ylmethylbenzene, 1,4-diamino-N-thiophen-3-ylmethylbenzene, 1,4-diamino-N-benzylbenzene, 1,4-diamino-2-(1-hydroxyethyl)benzene, 1,4-diamino-2-(2-hydroxyethyl)benzene, 1,4-diamino-2-(1-methylethyl)benzene, 1,3-bis[(4-aminophenyl)(2-hydroxyethyl)amino]-2-propanol, 1,4-bis[(4-aminophenyl)amino]butane, 1,8-bis(2,5-diaminophenoxy)-3,6-dioxaoctane, 2,5-diamino-4′-hydroxy-1,1′-biphenyl, 2,5-diamino-2′-trifluoromethyl-1,1′-biphenyl, 2,4′,5-triamino-1,1′-biphenyl, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-((4-methoxyphenyl)methyl-1H-pyrazole, 2-aminophenol, 2-amino-6-methylphenol, 2-amino-5-methylphenol, 1,2,4-trihydroxybenzene, 2,4-diaminophenol, 1,4-dihydroxy-benzene, 2-(((4-aminophenyl)amino)methyl)-1,4-diaminobenzene.

Furthermore, in addition to the compounds of the formula (I), the colorant according to the invention can also comprise further known coupler substances, for example N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxy-benzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 1,3-diamino-4-(3-hydroxypropoxy)benzene, 1,3-diamino-4-(2-methoxyethoxy)benzene, 2,4-diamino-1,5-di(2-hydroxyethoxy)benzene, 1-(2-aminoethoxy)-2,4-diaminobenzene, 2-amino-1-(2-hydroxyethoxy)-4-methyl-aminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxol, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione.

The additional coupler substances and developer substances may be present in the colorant according to the invention in each case individually or in the mixture with one another, where the total amount of coupler substances and developer substances in the colorant according to the invention (based on the total amount of the colorant) is in each case about 0.005 to 20 percent by weight, preferably about 0.01 to 5 percent by weight and in particular 0.1 to 2.5 percent by weight.

The total amount of the developer substance-coupler substance combination present in the colorant described here is preferably about 0.01 to 20 percent by weight, particular preference being given to an amount of from about 0.02 to 10 percent by weight and in particular 0.2 to 6 percent by weight. The developer substances and coupler substances are generally used in approximately equimolar amounts; however, it is not disadvantageous if the developer substances are present in this regard in a certain excess or deficit.

In addition, the colorant according to the invention can additionally comprise other color components, for example 6-amino-2-methylphenol and 2-amino-5-methylphenol, and also customary natural, nature-identical or synthetic direct dyes, for example triphenylmethane dyes, such as 4-[(4′-aminophenyl)(4′imino-2″,5″-cyclohexadien-1″-ylidene)methyl]-2-methylaminobenzene monohydrochloride (C.I. 42 510) and 4-[(4′-amino-3′-methylphenyl)(4″-imino-3″-methyl-2″,5″-cyclohexadien-1″-ylidene)methyl]-2-methylaminobenzene monohydrochloride (C.I. 42 520), aromatic nitro dyes, such as 4-(2′-hydroxyethyl)aminonitrotoluene, 2-amino-4,6-dinitrophenol, 2-amino-5-(2′-hydroxyethyl)aminonitrobenzene, 2-chloro-6-(ethylamino)-4-nitrophenol, 4-chloro-N-(2-hydroxyethyl-2-nitroaniline, 5-chloro-2-hydroxy-4-nitroaniline, 2-amino-4-chloro-6-nitrophenol or 1-[(2′-ureidoethyl)amino-4-nitrobenzene, azo dyes, such as 6-[(4′-aminophenyl)azo]-5-hydroxynaphthalene-1-sulfonic acid sodium salt (C.I. 14 805) or dispersion dyes, such as, for example, 1,4-diaminoanthraquinone and 1,4,5,8-tetraminoanthraquinone, and basic or acidic direct dyes. The colorant can comprise these color components in an amount of from about 0.1 to 4.0 percent by weight.

The coupler substances and developer substances and also the other color components, if they are bases, can of course also be used in the form of the physiologically compatible salts with organic or inorganic acids, such as, for example, hydrochloric acid or sulfuric acid, or—if they have aromatic OH groups—in the form of these salts with bases, for example as alkali metal phenoxides.

Moreover, if the colorants are to be used for coloring hair, they may also comprise further customary cosmetic additives, for example antioxidants, such as ascorbic acid, thioglycolic acid and sodium sulfite, and perfume oils, complexing agents, wetting agents, emulsifiers, thickeners and care substances. The preparation form of the colorant according to the invention can, for example, be a solution, in particular an aqueous or aqueous-alcoholic solution. The particularly preferred preparation forms are, however, a cream, a gel or an emulsion. Their composition is a mixture of the dye components with the additives customary for such preparations.

Customary additives in solutions, creams, emulsions or gels are, for example, solvents, such as water, lower aliphatic alcohols, for example ethanol, propanol or isopropanol, glycerol or glycols, such as 1,2-propylene glycol, and also wetting agents or emulsifiers from the classes of anionic, cationic, amphoteric or nonionogenic surface-active substances, such as, for example, fatty alcohol sulfates, oxyethylated fatty alcohol sulfates, alkylsulfonates, alkylbenzenesulfonates, alkyltrimethylammonium salts, alkylbetaines, oxyethylated fatty alcohols, oxyethylated nonylphenols, fatty acid alkanolamides and oxyethylated fatty acid esters, also thickeners, such as higher fatty alcohols, starch, cellulose derivatives, petrolatum, paraffin oil and fatty acids, and also care substances, such as cationic resins, lanolin derivatives, cholesterol, pantothenic acid and betaine. The constituents mentioned are used in the amounts customary for such purposes, for example the wetting agents and emulsifiers in concentrations of from about 0.5 to 30 percent by weight, the thickeners in an amount of from about 0.1 to 25 percent by weight and the care substances in a concentration of from about 0.1 to 5 percent by weight.

Depending on the composition, the colorant according to the invention can have a slightly acidic, neutral or alkaline reaction. In particular, it has a pH of from 6.8 to 11.5, the basic adjustment preferably being carried out with ammonia. It is, however, also possible to use organic amines, for example monoethanolamine and triethanolamine, and also inorganic bases, such as sodium hydroxide and potassium hydroxide. For a pH adjustment in the acidic range, inorganic or organic acids, for example phosphoric acid, acetic acid, citric acid or tartaric acid, are suitable.

For the use for the oxidative coloring of hair, the colorant described above is mixed directly prior to use with an oxidizing agent, and an amount of this mixture sufficient for the hair-coloring treatment, generally about 60 to 200 grams depending on the fullness of the hair, is applied to the hair.

Suitable oxidizing agents for developing the hair coloration are primarily hydrogen peroxide or its addition compounds onto urea, melamine, sodium borate or sodium carbonate in the form of a 3- to 12 percent strength, preferably 6 percent strength, aqueous solution, but also atmospheric oxygen. If a 6 percent strength hydrogen peroxide solution is used as oxidizing agent, then the weight ratio between hair colorant and oxidizing agent is 5:1 to 1:2, but preferably 1:1. Larger amounts of oxidizing agent are primarily used at higher dye concentrations in the hair colorant, or if stronger bleaching of the hair is intended at the same time.

The mixture is left to act on the hair at 15 to 50 degrees Celsius for about 10 to 45 minutes, preferably 30 minutes, then the hair is rinsed with water and dried. If required, this rinsing is followed by washing with a shampoo and possibly after-rinsing with a weak organic acid, such as, for example, citric acid or tartaric acid. The hair is then dried.

The hair colorants according to the invention with a content of 3-amino-2-aminomethylphenol derivatives of the formula (I) as coupler substances permit hair colorations with excellent color fastness, in particular with regard to light fastness, washing fastness and rubbing fastness. With regard to the coloring properties, the hair colorants according to the invention offer, depending on the nature and composition of the color components, a broad palette of different color nuances which ranges from blonde via brown, purple, violet to blue and black shades. The shades are characterized here by their particular color intensity. The very good coloring properties of the hair colorants according to the present application are further evident from the fact that these agents permit a coloring of gray, chemically non-predamaged hair without problems and with good coverage.

The present application further provides the 3-amino-2-aminomethylphenol derivatives of the formula (I) described above with the proviso that R1 and R4 are not hydrogen at the same time if (i) R2 and R3 are a methyl group or (ii) R2 and R3, together with the nitrogen atom, form a pyrrolidine ring or piperidine ring.

The aminophenol derivatives of the formula (I) according to the invention can be prepared using known synthesis methods. The synthesis of the compounds according to the invention can, for example, be carried out as follows:
either a) by a reductive amination of a substituted benzene of the formula (V) with subsequent reduction of the nitro group; 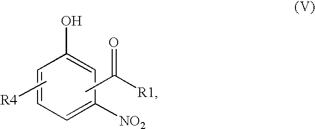
or b) by reduction of the nitrile group of the formula (VI), 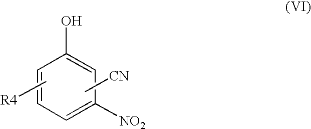
subsequent alkylation of the amino group with a compound of the formula XR2 and/or XR3, and final reduction of the nitro group, where R1, R2, R3 and R4 have the meanings given above and X is a halogen atom.

The 3-amino-2-aminomethylphenol derivatives of the formula (I) according to the invention are readily soluble in water and permit colorations with high color intensity and excellent color fastness, especially with regard to light fastness, washing fastness and rubbing fastness. The 3-amino-2-aminomethylphenol derivatives of the formula (I) also have excellent storage stability, especially as constituent of the colorants described above.

The examples below are intended to illustrate the subject-matter of the invention in more detail without limiting it thereto.

### EXAMPLES

### Examples 1 to 5

### Synthesis of 3-amino-2-aminomethylphenol Derivatives of the Formula (I)

### Synthesis of 3-amino-2-aminomethylphenol Derivatives (General Synthesis Procedure)

0.2 g (1.2 mmol) of 2-hydroxy-6-nitrobenzaldehyde and 1.8 mmol of the corresponding amine are dissolved in 6 ml of methanol. 0.25 ml of acetic acid and 0.15 g of NaBH₃CN (2.4 mmol) are then added and the reaction mixture is stirred for 1.5 to 5 hours at room temperature under argon in a sealed tube. When the reaction is complete, the reaction mixture is poured into 10 ml of ethyl acetate, and the organic phase is extracted with an aqueous solution of sodium hydrogen carbonate, then dried with magnesium sulfate and filtered. The solvent is distilled off on a rotary evaporator and the residue is purified over silica gel with petroleum ether/ethyl acetate (9:1). The product obtained in this way is hydrogenated with hydrogen in 4 ml of ethyl acetate in the presence of platinum oxide (20 mg). After 40 minutes, the mixture is filtered through Cellite and the filtrate is concentrated under reduced pressure. The product is produced in a yield of from 35 to 85%, in most cases in the form of an oil.

### 1. 3-Amino-2-(4-morpholinylmethyl)phenol

Amine used: morpholine

Yield: 0.212 g (85% of theory)

Mass spectrum: MH⁺ 209 (100)

### 2. 3-Amino-2-(1-pyrrolidinylmethyl)phenol

Amine used: pyrrolidine

Yield: 0.081 g (35% of theory)

Mass spectrum: MH⁺ 193 (100)

### 3. 3-Amino-2-{[(2-furylmethyl)amino]methyl]phenol

Amine used: 2-furylmethylamine

Yield: 0.165 g (63% of theory)

Mass spectrum: MH⁺ 219 (100)

### 4. 3-Amino-2-(1-piperidinylmethyl)phenol

Amine used: piperidine

Yield: 0.158 g (64% of theory)

Mass spectrum: MH⁺ 207 (100)

### 5. 3-Amino-2-[(4-methyl-1-piperazinyl)methyl]phenol

Amine used: 4-methylpiperazine

Yield: 0.122 g (46% of theory)

Mass spectrum: MH⁺ 222 (100)

### Examples 6 to 15

### Hair Colorants

Hair-coloring solutions of the following composition are prepared:

| | | |
|---|---|---|
| 1.25 | mmol | substance of the formula (I) as in table 1 |
| 1.25 | mmol | developer substance as in table 1 |
| 10.0 | g | lauryl ether sulfate (28 percent strength |
| | | aqueous solution) |
| 9.0 | g | ammonia (22 percent strength aqueous |
| | | solution) |
| 7.8 | g | ethanol |
| 0.3 | g | ascorbic acid |
| 0.3 | g | ethylenediaminotetraacetic acid disodium salt |
| | | hydrate |
| ad 100.0 | g | water, demineralized |

10 g of the above coloring solution are mixed directly prior to application with 10 g of a 6 percent strength hydrogen peroxide solution. The mixture is then applied to bleached hair. After a contact time of 30 minutes at 40° C., the hair is rinsed with water, washed with a standard commercial shampoo and dried. The resulting colorations are summarized in table 1.

TABLE 1

| | | Developer substance | | | | |
|---|---|---|---|---|---|---|
| | | | | III. | | |
| | | | | 4,5- | | |
| | | | II. | Diamino- | | V. |
| | | I. | 2,5- | 1-(2′- | | 2,4,5,6- |
| | Coupler | 2,5- | Diamino- | hydroxy- | | Tetra- |
| | substance | Diamino- | phenyl- | ethyl)- | IV. | amino- |
| Example | of the | toluene | ethanol | pyrazole | 4-Amino- | pyrimidine |
| No. | formula (I) | sulfate | sulfate | sulfate | phenol | sulfate |
| 6 | as in | violet | violet | intense | weak | blue-gray |
| | example 1 | | | raspberry | orange- | |
| | | | | red | brown | |
| 7 | as in | violet | violet | intense | weak | blue-gray |
| | example 2 | | | raspberry | orange- | |
| | | | | red | brown | |
| 8 | as in | dark violet | violet | intense | orange | gray |
| | example 3 | | | raspberry | | |
| | | | | red | | |
| 9 | as in | violet | violet | intense | weak | gray |
| | example 4 | | | raspberry | orange- | |
| | | | | red | brown | |
| 10 | as in | dark violet | violet | intense | red- | fennel |
| | example 5 | | | raspberry | brown | green |
| | | | | red | | |

Developer substance

| | Coupler | VI. | |
|---|---|---|---|
| | substance of | N,N-Bis(2-hydroxy- | VII. |
| Example | the formula | ethyl)-p-phenylene- | 4-Hydroxy-o- |
| No. | (I) | diamine | toluidine |
| 11 | as in | dark blue | pale red-brown |
| | example 1 | | |
| 12 | as in | dark blue | pale red-brown |
| | example 2 | | |
| 13 | as in | dark blue | pale red-brown |
| | example 3 | | |
| 14 | as in | dark blue | pale red-brown |
| | example 4 | | |
| 15 | as in | dark blue | pale red-brown |
| | example 5 | | |

### Examples 16 to 39

### Hair Colorants

Hair-coloring solutions of the following composition are prepared:
X g 3-aminophenol derivative of the formula (I) (coupler substance K1 to K5 as in table 4)
U g developer substance E8 to E15 as in table 2
Y g coupler substance K12 to K36 as in table 4
10.0 g lauryl ether sulfate (28 percent strength aqueous solution)
9.0 g ammonia (22 percent strength aqueous solution)
7.8 g ethanol
0.3 g ascorbic acid
0.3 g ethylenediaminotetraacetic acid disodium salt hydrate
ad 100.0 g water, demineralized

30 g of the above coloring solution are mixed directly prior to application with 30 g of a 6 percent strength aqueous hydrogen peroxide solution. The mixture is then applied to bleached hair. After a contact time of 30 minutes at 40° C., the hair is rinsed with water, washed with a standard commercial shampoo and dried. The coloring results are summarized in table 5.

TABLE 2
Developer substances

| | |
|---|---|
| E8 | 1,4-Diaminobenzene |
| E9 | 2,5-Diaminophenylethanol sulfate |
| E10 | 3-Methyl-4-aminophenol |
| E11 | 4-Amino-2-aminomethylphenol dihydrochloride |
| E13 | N,N-Bis(2′-hydroxyethyl)-p-phenylendiamine sulfate |
| E14 | 4,5-Diamino-1-(2′-hydroxyethyl)pyrazole sulfate |
| E15 | 2,5-Diaminotoluene sulfate |

TABLE 3
Direct dyes

| | |
|---|---|
| D2 | 6-Chloro-2-ethylamino-4-nitrophenol |
| D3 | 2-Amino-6-chloro-4-nitrophenol |

TABLE 4
Coupler substances

| | |
|---|---|
| K1 | 3-Amino-2-(4-morpholinylmethyl)phenol |
| K2 | 3-Amino-2-(1-pyrrolidinylmethyl)phenol |
| K3 | 3-Amino-2-{[(2-furylmethyl)amino]methyl}phenol |
| K4 | 3-Amino-2-(1-piperidinylmethyl)phenol |
| K5 | 3-Amino-2-[(4-methyl-1-piperazinyl)methyl]phenol |
| K12 | 2-Amino-4-(2′-hydroxyethyl)aminoanisole sulfate |
| K13 | 1,3-Diamino-4-(2′-hydroxyethoxy)benzene sulfate |
| K14 | 2,4-Diamino-5-fluorotoluene sulfate |
| K18 | N-(3-Dimethylamino)phenylurea |
| K19 | 1,3-Bis(2,4-diaminophenoxy)propane tetrahydrochloride |
| K21 | 3-Aminophenol |
| K22 | 5-Amino-2-methylphenol |
| K23 | 3-Amino-2-chloro-6-methylphenol |
| K24 | 5-Amino-4-fluoro-2-methylphenol sulfate |
| K25 | 1-Naphthol |
| K31 | 1,3-Dihydroxybenzene |
| K32 | 2-Methyl-1,3-dihydroxybenzene |
| K33 | 1-Chloro-2,4-dihydroxybenzene |
| K34 | 4-(2′-Hydroxyethyl)amino-1,2-methylenedioxybenzene |
| | hydrochloride |
| K36 | 2-Amino-5-methylphenol |

TABLE 5
Hair colorants

| | Example No. | | | | | |
|---|---|---|---|---|---|---|
| | 16 | 17 | 18 | 19 | 20 | 21 |
| Dyes | (amount of dye in grams) | | | | | |
| K1 | 0.10 | 0.12 | 0.05 | 0.07 | 0.10 | 0.12 |
| E8 | 0.30 | | | | | |
| E9 | | | | | 0.25 | 0.20 |
| E10 | | | | | | 0.10 |
| E15 | | 0.25 | 0.30 | 0.25 | | |
| K12 | | | 0.05 | | | |
| K13 | | | | 0.05 | | |
| K21 | 0.05 | | | | | |
| K22 | | 0.05 | | | | |
| K23 | | | 0.05 | 0.10 | 0.10 | 0.10 |
| K25 | | | | | 0.10 | |
| K31 | 0.20 | | | 0.15 | 0.10 | 0.10 |
| K32 | | 0.20 | | 0.10 | | |
| K33 | | | 0.20 | | | |
| K36 | | | | | | 0.10 |
| Coloring result | blonde | blonde | blonde | blonde | blonde | blonde |
| | Example No. | | | | | |
| | 22 | 23 | 24 | 25 | 26 | 27 |
| Dyes | (amount of dye in grams) | | | | | |
| K2 | 0.10 | 0.12 | 0.05 | 0.07 | 0.10 | 0.12 |
| E8 | 0.30 | | | | | |
| E9 | | | | | 0.25 | 0.20 |
| E10 | | | | | | 0.10 |
| E15 | | 0.25 | 0.30 | 0.25 | | |
| K12 | | | 0.05 | | | |
| K13 | | | | 0.05 | | |
| K21 | 0.05 | | | | | |
| K22 | | 0.05 | | | | |
| K23 | | | 0.05 | 0.10 | 0.10 | 0.10 |
| K25 | | | | | 0.10 | |
| K31 | 0.20 | | | 0.15 | 0.10 | 0.10 |
| K32 | | 0.20 | | 0.10 | | |
| K33 | | | 0.20 | | | |
| K36 | | | | | | 0.10 |
| Coloring result | blonde | blonde | blonde | blonde | blonde | blonde |
| | Example No. | | | | | |
| | 28 | 29 | 30 | 31 | 32 | 33 |
| Dyes | (amount of dye in grams) | | | | | |
| K3 | 0.10 | 0.12 | 0.05 | 0.07 | 0.10 | 0.12 |
| E8 | 0.30 | | | | | |
| E9 | | | | | 0.25 | 0.20 |
| E10 | | | | | | 0.10 |
| E15 | | 0.25 | 0.30 | 0.25 | | |
| K12 | | | 0.05 | | | |
| K13 | | | | 0.05 | | |
| K21 | 0.05 | | | | | |
| K22 | | 0.05 | | | | |
| K23 | | | 0.05 | 0.10 | 0.10 | 0.10 |
| K25 | | | | | 0.10 | |
| K31 | 0.20 | | | 0.15 | 0.10 | 0.10 |
| K32 | | 0.20 | | 0.10 | | |
| K33 | | | 0.20 | | | |
| K36 | | | | | | 0.10 |
| Coloring result | blonde | blonde | blonde | blonde | blonde | blonde |
| | Example No. | | | | | |
| | 34 | 35 | 36 | 37 | 38 | 39 |
| Dyes | (amount of dye in grams) | | | | | |
| K4 | 0.10 | 0.06 | 0.05 | | 0.10 | 0.12 |
| K5 | | 0.06 | | 0.07 | | |
| E8 | 0.30 | | | | | |
| E9 | | | | | 0.25 | 0.20 |
| E10 | | | | | | 0.10 |
| E15 | | 0.25 | 0.30 | 0.25 | | |
| K12 | | | 0.05 | | | |
| K13 | | | | 0.05 | | |
| K21 | 0.05 | | | | | |
| K22 | | 0.05 | | | | |
| K23 | | | 0.05 | 0.10 | 0.10 | 0.10 |
| K25 | | | | | 0.10 | |
| K31 | 0.20 | | | 0.15 | 0.10 | 0.10 |
| K32 | | 0.20 | | 0.10 | | |
| K33 | | | 0.20 | | | |
| K36 | | | | | | 0.10 |
| Coloring result | blonde | blonde | blonde | blonde | blonde | blonde |

### Examples 40 to 63

### Hair Colorants

Hair-coloring solutions of the following composition are prepared:
X g 3-aminophenol derivative of the formula (I) (coupler substance K1 to K4 as in table 4)
U g developer substance E8 to E15 as in table 2
Y g coupler substance K12 to K34 as in table 4
Z g direct dyes D2 and/or D3 as in table 3
10.0 g lauryl ether sulfate (28 percent strength aqueous solution)
9.0 g ammonia (22 percent strength aqueous solution)
7.8 g ethanol
0.3 g ascorbic acid
0.3 g ethylenediaminotetraacetic acid disodium salt hydrate
ad 100.0 g water, demineralized

30 g of the above coloring cream are mixed directly prior to application with 30 g of a 6 percent strength aqueous hydrogen peroxide solution. The mixture is then applied to the hair. After a contact time of 30 minutes at 40° C., the hair is rinsed with water, washed with a standard commercial shampoo and dried. The coloring results are summarized in table 6.

TABLE 6
Hair colorants

| | Example No. | | | | | |
|---|---|---|---|---|---|---|
| | 40 | 41 | 42 | 43 | 44 | 45 |
| Dyes | (amount of dye in grams) | | | | | |
| K1 | 0.60 | 1.30 | 1.15 | 0.15 | 0.15 | 0.15 |
| E8 | 1.50 | | | | | |
| E11 | 0.10 | | | | | |
| E13 | | 1.60 | | | | 0.70 |
| E14 | | | | 0.10 | 0.10 | |
| E15 | | | 1.80 | 0.70 | 0.70 | |
| K12 | 0.50 | | | | | |
| K14 | 0.10 | | | | | |
| K18 | 0.05 | | | | | |
| K19 | 0.10 | | | | | |
| K23 | | | 0.05 | 0.10 | 0.10 | 0.10 |
| K24 | 0.15 | | | | | |
| K31 | 0.90 | 1.10 | 1.10 | 0.40 | 0.40 | 0.40 |
| K34 | 0.10 | | | | | |
| D2 | | | | 0.10 | 0.10 | 0.10 |
| D3 | | | | 0.05 | 0.05 | 0.05 |
| Coloring result | black | black | black | brown | brown | brown |
| | Example No. | | | | | |
| | 46 | 47 | 48 | 49 | 50 | 51 |
| Dyes | (amount of dye in grams) | | | | | |
| K2 | 0.60 | 1.30 | 1.15 | 0.15 | 0.15 | 0.15 |
| E8 | 1.50 | | | | | |
| E11 | 0.10 | | | | | |
| E13 | | 1.60 | | | | 0.70 |
| E14 | | | | 0.10 | 0.10 | |
| E15 | | | 1.80 | 0.70 | 0.70 | |
| K12 | 0.50 | | | | | |
| K14 | 0.10 | | | | | |
| K18 | 0.05 | | | | | |
| K23 | | | 0.05 | 0.10 | 0.10 | 0.10 |
| K24 | 0.15 | | | | | |
| K31 | 0.90 | 1.10 | 1.10 | 0.40 | 0.40 | 0.40 |
| K34 | 0.10 | | | | | |
| D2 | | | | 0.10 | 0.10 | 0.10 |
| D3 | | | | 0.05 | 0.05 | 0.05 |
| Coloring result | black | black | black | brown | brown | brown |
| | Example No. | | | | | |
| | 52 | 53 | 54 | 55 | 56 | 57 |
| Dyes | (amount of dye in grams) | | | | | |
| K3 | 0.60 | 1.30 | 1.15 | 0.15 | 0.15 | 0.15 |
| E8 | 1.50 | | | | | |
| E11 | 0.10 | | | | | |
| E13 | | 1.60 | | | | 0.70 |
| E14 | | | | 0.10 | 0.10 | |
| E15 | | | 1.80 | 0.70 | 0.70 | |
| K12 | 0.50 | | | | | |
| K14 | 0.10 | | | | | |
| K18 | 0.05 | | | | | |
| K23 | | | 0.05 | 0.10 | 0.10 | 0.10 |
| K24 | 0.15 | | | | | |
| K31 | 0.90 | 1.10 | 1.10 | 0.40 | 0.40 | 0.40 |
| K34 | 0.10 | | | | | |
| D2 | | | | 0.10 | 0.10 | 0.10 |
| D3 | | | | 0.05 | 0.05 | 0.05 |
| Coloring result | black | black | black | brown | brown | brown |
| | Example No. | | | | | |
| | 58 | 59 | 60 | 61 | 62 | 63 |
| Dyes | (amount of dye in grams) | | | | | |
| K4 | 0.60 | 1.30 | 1.15 | 0.15 | 0.15 | 0.15 |
| E8 | 1.50 | | | | | |
| E11 | 0.10 | | | | | |
| E13 | | 1.60 | | | | 0.70 |
| E14 | | | | 0.10 | 0.10 | |
| E15 | | | 1.80 | 0.70 | 0.70 | |
| K12 | 0.50 | | | | | |
| K14 | 0.10 | | | | | |
| K18 | 0.05 | | | | | |
| K23 | | | 0.05 | 0.10 | 0.10 | 0.10 |
| K24 | 0.15 | | | | | |
| K31 | 0.90 | 1.10 | 1.10 | 0.40 | 0.40 | 0.40 |
| K34 | 0.10 | | | | | |
| D2 | | | | 0.10 | 0.10 | 0.10 |
| D3 | | | | 0.05 | 0.05 | 0.05 |
| Coloring result | black | black | black | brown | Brown | Brown |

Unless stated otherwise, all of the percentages given in the present application are percentages by weight.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An agent for the oxidative coloring of keratin fibers based on a developer substance-coupler substance combination which comprises at least one 3-amino-2-aminomethylphenol derivative of the formula (I) or its physiologically compatible, water-soluble salt, 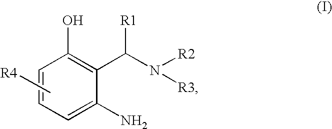
wherein
R1 is hydrogen or a C₁-C₆-alkyl group;
R2 and R3, independently of one another, are hydrogen, a saturated C₁-C₆-alkyl group, an unsaturated C₂-C₆-alkyl group, a C₂-C₄-hydroxyalkyl group, a C₃-C₄-dihydroxyalkyl group, a C₂-C₄-aminoalkyl group, a C₁-C₄-dimethylaminoalkyl group, a C₂-C₄-acetylaminoalkyl group, a C₁-C₄-methoxyalkyl group, a C₁-C₄-ethoxyalkyl group, a C₁-C₄-cyanoalkyl group, a C₁-C₄-carboxyalkyl group, a C₁-C₄-aminocarbonylalkyl group, a pyridylmethyl group, a furfuryl group, a hydrogenated furfuryl group, a substituted pyridyl group or a radical of the formula (II), (III) or (IV), 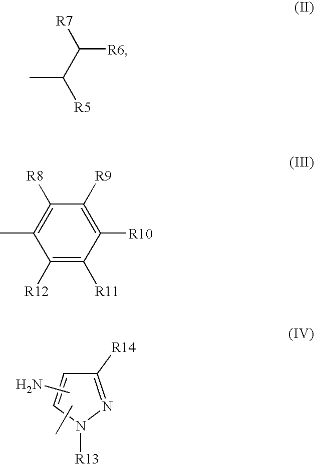
or R2 and R3, together with the nitrogen atom, form a heterocyclic ring of the formula; 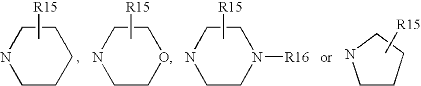
R4 is hydrogen, a halogen atom, a C₁-C₄-alkyl group, a C₁-C₄-hydroxyalkyl group or a C₁-C₄-alkoxy group;
R5 is hydrogen, a carboxyl group, or an aminocarbonyl group;
R6 and R7, independently of one another, are hydrogen, a hydroxyl group, an aminocarbonyl group, a methylthiomethyl group, a phenyl radical substituted by a phenyl group or hydroxyl group or a radical of the formula 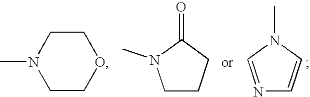
R8, R9, R10, R11 and R12, independently of one another, are hydrogen, a halogen atom, a cyano group, a hydroxyl group, a C₁-C₄-alkoxy group, a C₁-C₄-hydroxyalkoxy group, a C₁-C₆-alkyl group, a C₁-C₄-alkyl thioether group, a mercapto group, a nitro group, an amino group, an alkylamino group, a hydroxyalkylamino group, a dialkylamino group, a di(hydroxyalkyl)amino group, a (dihydroxyalkyl)amino group, a (hydroxyalkyl)alkylamino group, a trifluoromethane group, a —C(O)H— group, a —C(O)CH₃— group, a —C(O)CF₃— group, a —Si(CH₃)₃— group, a C₁-C₄-hydroxyalkyl group or a C₃-C₄-dihydroxyalkyl group or two adjacent radicals R8 to R12 form an —O—CH2-O— bridge;
R13 is a C₁-C₄-alkyl group or a C₁-C₄-hydroxyalkyl group;
R14 is hydrogen or a C₁-C₆-alkyl group;
R15 is one or more hydrogen atoms, hydroxyl groups, carboxyl groups, aminocarbonyl groups or hydroxymethyl groups;
R16 is hydrogen or a C₁-C₆-alkyl group; wherein at least one of the radicals R2 and R3 is different from hydrogen.

2. An agent according to claim 1, wherein the compound of the formula (I) is selected from the group consisting of compounds in which (i) R1 and/or R4 is hydrogen, and/or (ii) R2 is a methyl group or a C₁-C₄-hydroxyalkyl group and R3 is a C₁-C₄-hydroxyalkyl group and/or (iii) R2 is hydrogen and R3 is a C₁-C₄-hydroxyalkyl group, a substituted pyridyl radical, a furfuryl radical, a hydrogenated furfuryl radical, a substituted phenyl radical, a substituted pyrazoyl radical or a radical of the following formula 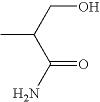
and/or (iv) R2 and R3 form an aliphatic ring of the formula

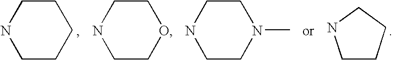

3. An agent according to claim 1, wherein the compound of the formula (I) is selected from the group consisting of 3-amino-2-[(2,3-dihydroxypropyl)-aminomethyl]phenol; 3-amino-2-[(2-aminoethylamino)methyl]phenol; 3-amino-2-[(2-hydroxyethylamino)-methyl]phenol; 3-amino-2-(4-morpholinylmethyl)phenol; 2-[(2-hydroxy-6-aminobenzyl)methylamino]ethanol; 3-amino-2-(1-pyrrolidinylmethyl)phenol, 3-amino-2-{[(2-furylmethyl)amino]methyl}phenol, 2-(2-hydroxy-6-amino-benzylamino)propan-1-ol; 2-[(2-hydroxy-6-aminobenzyl)-(2-hydroxyethyl)amino]ethanol; [1-(2-hydroxy-6-amino-benzyl)pyrrolidin-2-yl]methanol; 1-(2-hydroxy-6-aminobenzyl)pyrrolidine-2-carboxamide; 3-amino-2-[(4-methylpyridin-2-ylamino)methyl]phenol; 3-amino-2-(1-piperidinylmethyl)phenol, 3-amino-2-[(4-methyl-1-piperazinyl)methyl]phenol, 3-amino-2-((2-aminophenylamino)methyl)phenol; 3-amino-2-((2-chloro-phenylamino)methyl)phenol; 3-amino-2-[(2-dimethylaminophenylamino)methyl]phenol; 3-amino-2-[(2-fluorophenylamino)methyl]phenol; 3-amino-2-{[2-(2-hydroxyethylamino)phenyl-amino]methyl}phenol; 3-amino-2-{[2-N,N-bis(2-hydroxy-ethyl)aminophenylamino]methyl}phenol; 3-amino-2-[(2-pyrrolidinephenylamino)methyl)phenol; 3-amino-2-((3-aminophenylamino)methyl)phenol; 3-amino-2-[(3-chlorophenylamino)methyl]phenol; 3-amino-2-[(3-dimethylaminophenylamino)methyl]phenol; 3-amino-2-[(3-fluorophenyl-amino)methyl]phenol; 3-amino-2-{[3-(2-hydroxyethyl-amino)phenylamino]methyl}phenol; 3-amino-2-{[3-N,N-bis(2-hydroxyethyl)aminophenylamino]methyl}phenol; 3-amino-2-[(3-pyrrolidinephenylamino)methyl}phenol; 3-amino-2-[(4-aminophenylamino)methyl]phenol; 3-amino-2-((4-chlorophenylamino)methyl)phenol; 3-amino-2-[(4-dimethylaminophenylamino)methyl]phenol; 3-amino-2-[(4-fluorophenylamino)methyl]phenol; 3-amino-2-{[4-(2-hydroxyethylamino)phenylamino]methyl}phenol; 3-amino-2-{[4-N,N-bis(2-hydroxyethyl)aminophenylamino]methyl}phenol; 3-amino-2-[(4-pyrrolidinephenylamino)methyl]phenol; 3-amino-2-{[2-(2-hydroxyethoxy)-4-aminophenylamino]methyl}phenol; 3-amino-2-[(2,4-diaminophenyl-amino)methyl]phenol; 3-amino-2-[(2-chloro-4-aminophenylamino)methyl]phenol; 3-amino-2-[(4-amino-2-hydroxyphenylamino)methyl]phenol; 3-amino-2-[(2-hydroxyethylamino-4-aminophenylamino)methyl]phenol; 3-amino-2-[(2-methyl-4-aminophenylamino)methyl]phenol; 3-amino-2-{[3-(2-hydroxyethoxy)-4-aminophenylamino]methyl}phenol; 3-amino-2-[(3-amino-4-aminophenylamino)-methyl]phenol; 3-amino-2-[(3-chloro-4-aminophenylamino)methyl]phenol; 3-amino-2-[(3-hydroxy-4-aminophenylamino)methyl]phenol; 3-amino-2-{[3-(2-hydroxyethylamino)-4-aminophenylamino]methyl}phenol; 3-amino-2-[(3-methyl-4-aminophenylamino)methyl]phenol; 3-amino-2-{[2-(2-hydroxyethoxy)phenylamino]methyl}phenol; 3-amino-2-[(2-hydroxyphenylamino)methyl}phenol; 3-amino-2-{[3-(2-hydroxyethoxy)phenylamino]methyl}phenol; 3-amino-2-[(3-hydroxyphenylamino)methyl]phenol; 3-amino-2-{[4-(2-hydroxyethoxy)phenylamino]methyl}phenol; 3-amino-2-[(4-hydroxyphenylamino)methyl}phenol; 3-amino-2-(phenylaminomethyl)phenol and 2-[5-amino-4-(2-hydroxy-6-aminobenzylamino)pyrazol-1-yl]ethanol.

4. An agent according to claim 1, wherein the 3-amino-2-aminomethylphenol derivative of the formula (I) is present in an amount of from about 0.005 to 20 percent by weight.

5. An agent according to claim 1, wherein the developer substance is selected from the group consisting of 1,4-diaminobenzene (p-phenylenediamine), 1,4-diamino-2-methylbenzene (p-tolylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-((diethylamino)methyl)thiophene, 2-chloro-3-(2,5-diaminophenyl)thiophene, 1,4-diamino-2-(pyridin-3-yl)benzene, 2,5-diaminobiphenyl, 2,5-diamino-4′-(1-methylethyl)-1,1′-biphenyl, 2,3′,5-triamino-1,1′-biphenyl, 1,4-diamino-2-methoxymethylbenzene, 1,4-diamino-2-aminomethylbenzene, 1,4-diamino-2-((phenylamino)methyl)benzene, 1,4-diamino-2-((ethyl-(2-hydroxyethyl)amino)methyl)benzene, 1,4-diamino-2-hydroxymethylbenzene, 1,4-diamino-2-(2-hydroxyethoxy)benzene, 2-(2-(acetylamino)ethoxy)-1,4-diaminobenzene, 4-phenylaminoaniline, 4-dimethylamino-aniline, 4-diethylaminoaniline, 4-dipropylaminoaniline, 4-[ethyl(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 4-(((4-aminophenyl)methyl)amino)aniline, 4-[(4-aminophenylamino)methyl]phenol, 1,4-diamino-N-(4-pyrrolidin-1-yl-benzyl)benzene, 1,3-dihydroxy-2-((2-furylmethyl)aminomethyl)benzene, 1,4-diamino-N-thiophen-2-ylmethylbenzene, 1,4-diamino-N-furan-2-ylmethylbenzene, 1,4-diamino-N-thiophen-3-ylmethylbenzene, 1,4-diamino-N-benzylbenzene, 1,4-diamino-2-(1-hydroxyethyl)benzene, 1,4-diamino-2-(2-hydroxyethyl)benzene, 1,4-diamino-2-(1-methylethyl)benzene, 1,3-bis[(4-aminophenyl)(2-hydroxyethyl)amino]-2-propanol, 1,4-bis[(4-aminophenyl)amino]butane, 1,8-bis(2,5-diaminophenoxy)-3,6-dioxaoctane, 2,5-diamino-4′-hydroxy-1,1′-biphenyl, 2,5-diamino-2′-trifluoromethyl-1,1′-biphenyl, 2,4′,5-triamino-1,1′-biphenyl, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chloro-phenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-((4-methoxyphenyl)methyl-1H-pyrazole, 2-aminophenol, 2-amino-6-methylphenol, 2-amino-5-methylphenol, 1,2,4-trihydroxybenzene, 2,4-diaminophenol, 1,4-dihydroxybenzene, and 2-(((4-aminophenyl)amino)methyl)-1,4-diaminobenzene.

6. An agent according to claim 1, wherein the developer substances and coupler substances, based on the total amount of the colorant, are present in each case in a total amount of from about 0.005 to 20 percent by weight.

7. An agent according to claim 1, wherein the agent additionally comprises at least one additional coupler substance and/or at least one direct dye.

8. An agent according to claim 1, wherein the agent is a hair colorant.

9. A 3-Amino-2-aminomethylphenol derivative of the formula (I) or physiologically compatible, water-soluble salt thereof, 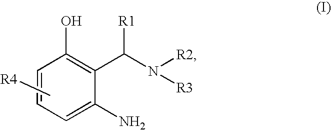
in which
R1 is hydrogen or a C₁-C₆-alkyl group;
R2 and R3, independently of one another, are hydrogen, a saturated C₁-C₆-alkyl group, an unsaturated C₂-C₆-alkyl group, a C₂-C₄-hydroxyalkyl group, a C₃-C₄-dihydroxyalkyl group, a C₂-C₄-aminoalkyl group, a C₁-C₄-dimethylaminoalkyl group, a C₂-C₄-acetylaminoalkyl group, a C₁-C₄-methoxyalkyl group, a C₁-C₄-ethoxyalkyl group, a C₁-C₄-cyanoalkyl group, a C₁-C₄-carboxyalkyl group, a C₁-C₄-aminocarbonylalkyl group, a pyridylmethyl group, a furfuryl group, a hydrogenated furfuryl group, a substituted pyridyl group or a radical of the formula (II), (III) or (IV), 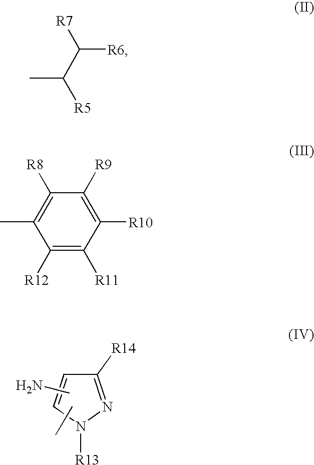
or R2 and R3, together with the nitrogen atom, form a heterocyclic ring of the formula; 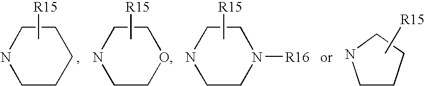
R4 is hydrogen, a halogen atom, a C₁-C₄-alkyl group, a C₁-C₄-hydroxyalkyl group or a C₁-C₄-alkoxy group;
R5 is hydrogen, a carboxyl group, or an aminocarbonyl group;
R6 and R7, independently of one another, are hydrogen, a hydroxyl group, an aminocarbonyl group, a methylthiomethyl group, a phenyl radical substituted by a phenyl group or hydroxyl group or a radical of the formula 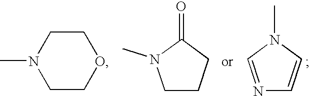
R8, R9, R10, R11 and R12, independently of one another, are hydrogen, a halogen atom, a cyano group, a hydroxyl group, a C₁-C₄-alkoxy group, a C₁-C₄-hydroxyalkoxy group, a C₁-C₆-alkyl group, a C₁-C₄-alkyl thioether group, a mercapto group, a nitro group, an amino group, an alkylamino group, a hydroxyalkylamino group, a dialkylamino group, a di(hydroxyalkyl)amino group, a (dihydroxyalkyl)amino group, a (hydroxyalkyl)alkylamino group, a trifluoromethane group, a —C(O)H— group, a —C(O)CH₃— group, a —C(O)CF₃— group, a —Si(CH₃)₃— group, a C₁-C₄-hydroxyalkyl group or a C₃-C₄-dihydroxyalkyl group or two adjacent radicals R8 to R12 form an —O—CH2-O-bridge;
R13 is a C₁-C₄-alkyl group or a C₁-C₄-hydroxyalkyl group;
R14 is hydrogen or a C₁-C₆-alkyl group;
R15 is one or more hydrogen atoms, hydroxyl groups, carboxyl groups, aminocarbonyl groups or hydroxymethyl groups;
R16 is hydrogen or a C₁-C₆-alkyl group; wherein at least one of the radicals R2 and R3 is different from hydrogen, and R1 and R4 are not hydrogen at the same time if R2 and R3 are a methyl group or R2 and R3, together with the nitrogen atom, form a pyrrolidine ring or piperidine ring.
